(19) United States
(12) Patent Application Publication
(10) Pub. No.: US 2017/0216295 A1
(43) Pub. Date: Aug. 3, 2017

(54) CCR9 ANTAGONIST COMPOUNDS

(71) Applicants: Bhuamik Pandya, Bedford, MA (US); Blaise Lippa, Acton, MA (US); Xin Zhang, Belmont, MA (US); Jon Christian Baber, Somerville, MA (US); Jan Antionette C. Romero, Somerville, MA (US); Jing Zhang, Lexington, MA (US)

(72) Inventors: Bhuamik Pandya, Bedford, MA (US); Blaise Lippa, Acton, MA (US); Xin Zhang, Belmont, MA (US); Jon Christian Baber, Somerville, MA (US); Jan Antionette C. Romero, Somerville, MA (US); Jing Zhang, Lexington, MA (US)

(73) Assignee: Merck Sharp & Dohme Corp., Rahway, NJ (US)

(21) Appl. No.: 15/501,103

(22) PCT Filed: Jul. 27, 2015
(86) PCT No.: PCT/US15/42160

§ 371 Date: Feb. 1, 2017

Related U.S. Application Data
(60) Provisional application No. 62/031,889, filed on Aug. 1, 2014.
(60) Provisional application No. 62/059,507, filed on Oct. 3, 2014.
(60) Provisional application No. 62/074,299, filed on Nov. 3, 2014.

(51) Int. Cl. A61K 31/519; C07D 487/04

(52) U.S. Cl. CPC A61K 31/519; C07D 487/04

(57) ABSTRACT

Provided herein are compounds that inhibit CCR9 receptor function. Also provided herein are methods of treating inflammatory disease in a subject, comprising administering to the subject a compound of the invention. Accordingly, in one aspect, provided herein is a compound of Formula (1) or a pharmaceutically acceptable salt thereof. In another aspect, provided herein is a pharmaceutical composition, comprising a compound of Formula (1), and a pharmaceutically acceptable carrier.

### BACKGROUND OF THE INVENTION

Inflammatory bowel disease (IBD), including Crohn's disease (CD) and ulcerative colitis (UC), is characterized by chronic inflammation in the gastrointestinal (GI) tract with periods of flare and remission. Steroids and immuno-modulators are widely used treatments but are sub-optimal in their effectiveness. In the last decade, the standard of care in the US has become anti-TNFa monoclonal antibodies (e.g. infliximab) in combination with the immunosuppressant azathioprine which benefits two thirds of CD patients. Of the patients that do respond, ˜50% remain in remission after a year of therapy. Thus, there remains a significant unmet medical need for those that either did not respond initially or did not maintain their remission after one year.

### SUMMARY OF INVENTION

Provided herein are compounds that inhibit CCR9 receptor function. Also provided herein are methods of treating inflammatory disease in a subject, comprising administering to the subject a compound of the invention.

Accordingly, in one aspect, provided herein is a compound of Formula I:

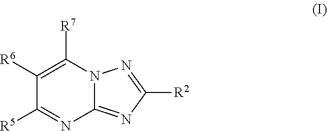

or a pharmaceutically acceptable salt thereof.

In another aspect, provided herein is a pharmaceutical composition, comprising a compound of Formula I, and a pharmaceutically acceptable carrier.

In still another aspect, provided herein is a method of treating an inflammatory disease in a subject in need thereof, comprising administering to the subject an effective amount of a compound of Formula I, or a pharmaceutically acceptable salt thereof. In one embodiment, the inflammatory disease is inflammatory bowel disease. In another embodiment, the inflammatory disease is Crohn's disease or ulcerative colitis.

In another aspect, provided herein is a method of inhibiting a CCR9 receptor function in a subject in need thereof, comprising the step of administering to the subject an effective amount of a compound of Formula I, or a pharmaceutically acceptable salt thereof. In one embodiment, the compound inhibits the binding of a ligand to CCR9. In another embodiment, the ligand is TECK.

In another aspect, provided herein is a method of inhibiting CCR9-mediated homing of leukocytes in a subject in need of such treatment, comprising administering to the subject an effective amount of a compound of Formula I, or a pharmaceutically acceptable salt thereof.

### BRIEF DESCRIPTION OF DRAWINGS

FIG. 1a shows the pharmacokinetic profiles of selected compounds of the invention.

FIG. 1b shows the pharmacokinetic profiles of selected compounds of the invention.

FIG. 2 shows that the oral bioavailability of selected compounds of the invention was found to be highly sensitive to their physicochemical properties such as pH (Ref Solubility, pH units), solubility (mg/ml) and precipitation time (sec).

FIG. 3 shows the oral pharmacokinetic profiles of selected compounds of the invention.

FIG. 4 shows clearance structure activity relationships in selected compounds of the invention.

FIG. 5 shows clearance structure activity relationships in selected compounds of the invention with correction for plasma and microsome protein binding.

### DETAILED DESCRIPTION OF THE INVENTION

The GI tract inflammation associated with IBD results from inappropriate recruitment and accumulation of leukocytes in the gut. CCR9 is a key mediator for pro-inflammatory T cells to migrate from the blood stream to the gut tissue. The CCR9 ligand (CCL25) is expressed predominantly in the thymus and the small intestine. In CD patients, chemokine CCL25 is overexpressed in the small intestine and CCR9+ lymphocytes are reported to be significantly elevated.

Provided herein are compounds according to Formulae I, II, III, IV and V. Also provided herein are CCR9 inhibitors for the treatment of inflammatory diseases comprising administering to the subject a compound provided herein. Also provided herein are methods of treating an inflammatory disease in a subject in need thereof comprising administering to the subject a compound provided herein. Also provided herein are methods of inhibiting a CCR9 receptor function in a subject in need thereof comprising administering to the subject a compound provided herein. Also provided herein are methods of inhibiting CCR9-mediated homing of leukocytes in a subject in need of such treatment comprising administering to the subject a compound provided herein. In some embodiments, the inflammatory disease is inflammatory bowel disease, Crohn's disease, or ulcerative colitis. In other embodiments, the compound of Formulae I, II, III, IV or V inhibits the binding of a ligand to CCR9. In an embodiment, the ligand is TECK.

In one aspect, provided herein is a compound of Formula I:

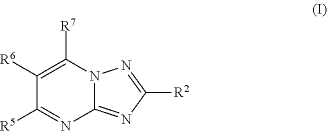

or a pharmaceutically acceptable salt thereof,

wherein:

R² is H, C₁₋₆ alkyl, C₁₋₆ alkoxy optionally substituted one or more times with OH, C₁₋₆ haloalkyl, C₁₋₆ di-haloalkyl, C₁₋₆ tri-haloalkyl, NH₂, N(H)(C₁₋₃ alkyl), N(C₁₋₃ alkyl)₂, (CH₂)₁₋₄7 NH₂, (CH₂)₁₋₄—N(H)(C₁₋₃ alkyl), (CH₂)₁₋₄—N(C₁₋₃ alkyl)₂, (CH₂)₁₋₄—C₁₋₆ alkoxy, C(O)NH₂, C(O)N(H)(C₁₋₃ alkyl), C(O)N(C₁₋₃ alkyl)₂, OH, (CH₂)₁₋₄—OH, or a C₃₋₅ heterocycle optionally substituted one or more times with OH;

R⁵ is OH, C₁₋₆ alkyl, C₁₋₆ alkoxy, (CH₂)₁₋₄—C₁₋₆ alkoxy, C₃₋₇ cycloalkyl, N(C₁₋₃ alkyl)₂, or heterocycle;

R⁶ is (CH₂)₁₋₄-aryl, wherein aryl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and

R⁷ is OH, C₁₋₃ alkyl, or C₁₋₃ alkoxy.

In another embodiment of Formula I, R² is H, CF₃, or (CH₂)₁₋₄—C₁₋₆ alkoxy.

In another embodiment of Formula I, R² is H.

In another embodiment of Formula I, R⁵ is C₁₋₆ alkyl, (CH₂)₁₋₄—C₁₋₆ alkoxy, C₃₋₇ cycloalkyl, heterocycle, OH, NH₂, N(H)(C₁₋₃ alkyl), or N(C₁₋₃ alkyl)₂.

In another embodiment of Formula I, R⁵ is C₁₋₆ alkyl.

In another embodiment of Formula I, R⁷ is CH₃ or OH.

In another embodiment of Formula I, R⁶ is (CH₂)₁₋₄-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy.

In another embodiment of Formula I, R⁶ is (CH₂)-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy.

In another embodiment of Formula I, R⁶ is (CH₂)-phenyl, wherein phenyl can be optionally independently substituted one or more times with C₁₋₆ alkyl or C₁₋₆ alkoxy, wherein the C₁₋₆ alkyl group is optionally substituted with CN.

In another embodiment of Formula I:

R² is H, C₁₋₆ alkyl, CF₃, NH₂, N(H)(C₁₋₃ alkyl), N(C₁₋₃ alkyl)₂, (CH₂)₁₋₄—NH₂₅ (CH₂)₁₋₄7 N(H)(C₁₋₃ alkyl), (CH₂)₁₋₄—N(C₁₋₃ alkyl)₂, (CH₂)₁₋₄—C₁₋₆ alkoxy, C(O)N(C₁₋₃ alkyl)₂, or (CH₂)₁₋₄—OH;

R⁵ is OH, C₁₋₆ alkyl, C₁₋₆ alkoxy, (CH₂)₁₋₄—C₁₋₆ alkoxy, C₃₋₇ cycloalkyl, or heterocycle;

R⁶ is (CH₂)₁₋₄-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and

R⁷ is OH.

In another embodiment of Formula I:

R² is H, C₁₋₆ alkyl, C₁₋₆ alkoxy, C₁₋₆ haloalkyl, C₁₋₆ di-haloalkyl, C₁₋₆ tri-haloalkyl, NH₂, N(H)(C₁₋₃ alkyl), N(C₁₋₃ alkyl)₂, (CH₂)₁₋₄—NH₂, (CH₂)₁₋₄—N(H)(C₁₋₃ alkyl), (CH₂)₁₋₄—N(C₁₋₃ alkyl)₂, (CH₂)₁₋₄—C₁₋₆ alkoxy, C(O)NH₂, C(O)N(H)(C₁₋₃ alkyl), C(O)N(C₁₋₃ alkyl)₂, OH, or (CH₂)₁₋₄—OH;

R⁵ is OH, C₁₋₆ alkyl, C₁₋₆ alkoxy, (CH₂)₁₋₄—C₁₋₆ alkoxy, C₃₋₇ cycloalkyl, N(C₁₋₃ alkyl)₂, or heterocycle;

R⁶ is (CH₂)₁₋₄-aryl, wherein aryl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and

R⁷ is OH, C₁₋₃ alkyl, or C₁₋₃ alkoxy.

In one embodiment of Formula I, R² is H, CF₃, or (CH₂)₁₋₄—C₁₋₆ alkoxy, or C₁₋₆ alkoxy optionally substituted with OH.

In one embodiment of Formula I, R² is H, C₁₋₆ alkyl, CF₃, NH₂, N(H)(C₁₋₃ alkyl), N(C₁₋₃ alkyl)₂, (CH₂)₁₋₄—NH₂, (CH₂)₁₋₄—N(H)(C₁₋₃ alkyl), (CH₂)₁₋₄—N(C₁₋₃ alkyl)₂, (CH₂)₁₋₄—C₁₋₆ alkoxy, C(O)N(C₁₋₃ alkyl)₂, or (CH₂)₁₋₄—OH, or C₁₋₆ alkoxy optionally substituted with OH;

R⁵ is OH, C₁₋₆ alkyl, C₁₋₆ alkoxy, (CH₂)₁₋₄—C₁₋₆ alkoxy, C₃₋₇ cycloalkyl, or heterocycle;

R⁶ is (CH₂)₁₋₄-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and

R⁷ is OH.

In another embodiment, the compound of Formula I is selected from the compounds of Table 1, or pharmaceutically acceptable salts thereof.

In another embodiment, the compound of Formula I is selected from the compounds of Table 2, or pharmaceutically acceptable salts thereof.

In another embodiment, the compound of Formula I is selected from the compounds of Table 3, or pharmaceutically acceptable salts thereof.

In another embodiment, the compound of Formula I is selected from the compounds of Table 4, or pharmaceutically acceptable salts thereof.

In another embodiment, provided herein is a pharmaceutical composition comprising a compound of Formula I and a pharmaceutically acceptable carrier.

In another aspect, provided herein is a method of treating an inflammatory disease in a subject in need thereof, comprising administering to the subject an effective amount of a compound of Formula I. In one embodiment, the inflammatory disease is inflammatory bowel disease. In another embodiment, the inflammatory disease is Crohn's disease or ulcerative colitis.

In yet another aspect, provided herein is a method of inhibiting CCR9 receptor function in a subject in need thereof, comprising the step of administering to the subject an effective amount of a compound of Formula I. In one embodiment, the compound inhibits the binding of a ligand to CCR9. In yet another embodiment, the ligand is TECK.

In still another aspect, provided herein is a method of inhibiting CCR9-mediated homing of leukocytes in a subject in need of such treatment, comprising administering to the subject an effective amount of at least one compound of Formula I.

In another aspect, provided herein is a compound of Formula II:

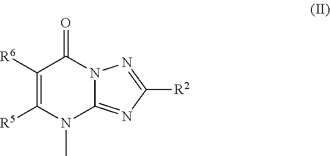

or a pharmaceutically acceptable salt thereof, wherein variables R², R⁵, and R⁶ have the definitions provided for Formula I.

In another aspect, provided herein is a compound of Formula III:

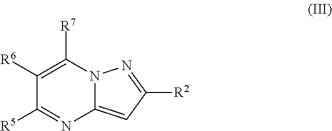

or a pharmaceutically acceptable salt thereof, wherein variables R², R⁵, R⁶, and R⁷ have the definitions provided for Formula I.

In another aspect, provided herein is a compound of Formula IV:

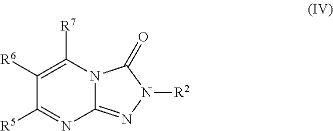

or a pharmaceutically acceptable salt thereof, wherein variables R², R⁵, R⁶, and R⁷ have the definitions provided for Formula I.

In another aspect, provided herein is a compound of Formula V:

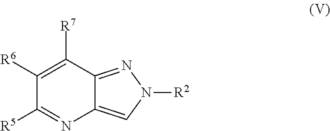

or a pharmaceutically acceptable salt thereof, wherein variables R², R⁵, R⁶, and R⁷ have the definitions provided for Formula I.

Representative compounds of Formulae I, II, III, IV and V include, but are not limited to, the following compounds of Table 1 below, or pharmaceutically acceptable salts thereof

TABLE 1

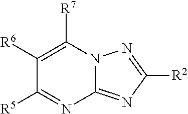

| | | | | | CCR9 | | Chemot- | RLM/HL | |
|---|---|---|---|---|---|---|---|---|---|
| | | | | | Ca²⁺ | | axis | M CL^{†} | Sol. pH |
| | | | | | IC₅₀ | Lip | (buffer, | (mL/min/ | 4/7/9^{‡} |
| ID | Core | R² | R⁵ | R⁶ | (uM) | E* | uM) | kg) | (ug/mL) |
| 1 | 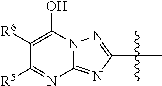 | CF₃ | Me | 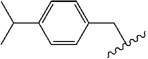 [SMILES: *Cc1ccc(C(C)C)cc1] | 0.73 | 3.5 | — | 51/19 | |
| 2 | 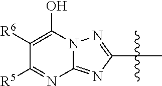 | CF₃ | Me | 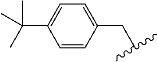 [SMILES: *Cc1ccc(C(C)(C)C)cc1] | 0.41 | 2.6 | — | 54/19 | 4/565/1035 |
| 3 | 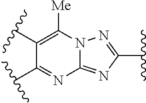 [SMILES: *c1nc2nc(*)c(*)c(C)n2n1] | H | OH | 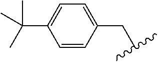 [SMILES: *Cc1ccc(C(C)(C)C)cc1] | 0.82 | 3.7 | — | — | — |
| 4 | 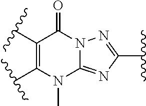 [SMILES: *c1nc2n(C)c(*)c(*)c(=O)n2n1] | CF₃ | Me | 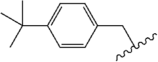 [SMILES: *Cc1ccc(C(C)(C)C)cc1] | >20 | — | — | — | — |
| 5 | 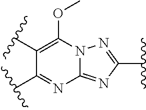 [SMILES: *c1nc2nc(*)c(*)c(OC)n2n1] | CF₃ | Me | 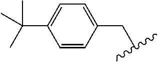 [SMILES: *Cc1ccc(C(C)(C)C)cc1] | 3.7 | 0.1 | — | — | — |
| 6 | 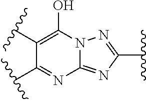 [SMILES: *c1nc2nc(*)c(*)c(O)n2n1] | H | Me | 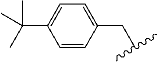 [SMILES: *Cc1ccc(C(C)(C)C)cc1] | 1.1 | 2.4 | — | — | — |
| 7 | 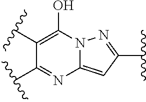 [SMILES: *c1cc2nc(*)c(*)c(O)n2n1] | CF₃ | Me | 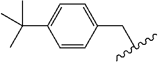 [SMILES: *Cc1ccc(C(C)(C)C)cc1] | 0.11 | 2.6 | 0.98 | 42/18 | 0/0/5 |
| 8 | 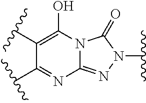 [SMILES: *c1nc2nn(*)c(=O)n2c(O)c1*] | H | Et | 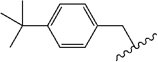 [SMILES: *Cc1ccc(C(C)(C)C)cc1] | 0.028 | 5.5 | 0.23 | 39/4 | 79/117/914 |
| 9 | 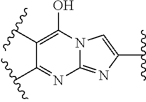 [SMILES: *c1cn2c(O)c(*)c(*)nc2n1] | Me | Me | 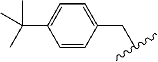 [SMILES: *Cc1ccc(C(C)(C)C)cc1] | >10 | — | — | — | — |
| 10 | 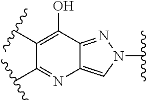 [SMILES: *c1nc2cn(*)nc2c(O)c1*] | H | Et | 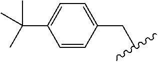 [SMILES: *Cc1ccc(C(C)(C)C)cc1] | >10 | — | — | — | — |
| 39 | 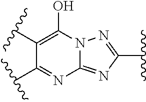 [SMILES: *c1nc2nc(*)c(*)c(O)n2n1] | 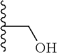 [SMILES: *CO] | Me | 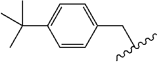 [SMILES: *Cc1ccc(C(C)(C)C)cc1] | | | | | |
| 40 | 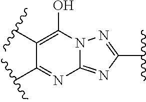 [SMILES: *c1nc2nc(*)c(*)c(O)n2n1] | 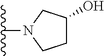 [SMILES: *N1CC[C@@H](O)C1] | Et | 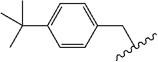 [SMILES: *Cc1ccc(C(C)(C)C)cc1] | | | | | |
| 41 | 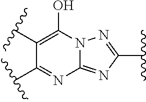 [SMILES: *c1nc2nc(*)c(*)c(O)n2n1] | 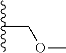 [SMILES: *COC] | Me | 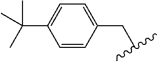 [SMILES: *Cc1ccc(C(C)(C)C)cc1] | | | | | |
| 42 | 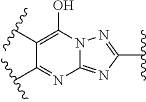 [SMILES: *c1nc2nc(*)c(*)c(O)n2n1] | 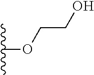 [SMILES: *OCCO] | Me | 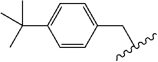 [SMILES: *Cc1ccc(C(C)(C)C)cc1] | | | | | |

*LipE = pIC₅₀-clogD;
^{†}RLM/HLM CL = rat liver microsome/human liver microsome hepatic clearance;
^{‡}Sol. pH 4/7/9 = Solubility of 1000 ug compound in 1 mL pH 4/7/9 buffer

Representative compounds of Formula I include, but are not limited to, the following compounds of Table 2 below, or pharmaceutically acceptable salts thereof

TABLE 2

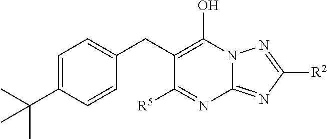

| | | | CCR9 Ca²⁺ | | Chemotaxis | RLM/HLM CL | Sol. pH 4/7/9 |
|---|---|---|---|---|---|---|---|
| ID | R² | R⁵ | IC₅₀ (uM) | LipE | (buffer, uM) | (mL/min/kg) | (ug/mL) |
| 11 | H | Me | 0.18 | 4.4 | 0.48 | 51/19 | 13/136/>1000 |
| 12 | Me | Me | 0.22 | 3.9 | — | 50/11 | — |
| 13 | Me | Et | 0.019 | 4.4 | 0.13 | — | — |
| 14 | NH₂ | Et | 0.002 | 5.9 | 0.050 | 39/6 | 2/7/358 |
| 15 | NMe₂ | Et | 0.033 | 4.1 | 0.38 | 43/12 | — |
| 16 | CH₂NMe₂ | Et | 0.29 | 3.1 | — | — | — |
| 17 | CH₂OCH₃ | Et | 0.007 | 5.2 | 0.19 | 36/13 | — |
| 18 | CONMe₂ | Et | 0.012 | 5.2 | 0.52 | 42/18 | 0/0/5 |
| 19 | OEt | Et | 0.008 | 4.2 | 0.15 | 16/11 | 79/117/914 |
| 20 | CH₂OH | Et | 0.010 | 5.5 | 0.20 | 43/8 | — |

Representative compounds of Formula I include, but are not limited to, the following compounds of Table 3 below, or pharmaceutically acceptable salts thereof.

TABLE 3

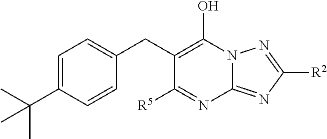

| | | | CCR9 Ca²⁺ | Lip | Chemotaxis | RLM/HLM CL | Sol. pH 4/7/9 |
|---|---|---|---|---|---|---|---|
| ID | R² | R⁵ | IC₅₀ (uM) | E | (buffer, uM) | (mL/min/kg) | (ug/mL) |
| 11 | H | Me | 0.18 | 4.4 | 0.48 | 51/19 | 13/136/>1000 |
| 21 | H | Et | 0.021 | 4.8 | 0.088 | 51/12 | — |
| 22 | H | nPr | 0.004 | 5.0 | 0.055 | — | — |
| 23 | H | iPr | 0.052 | 4.0 | 0.34 | 44/13 | 2/7/358 |
| 24 | H | iBu | 0.027 | 3.9 | 0.27 | 43/12 | — |
| 25 | H | OEt | 0.023 | 4.8 | 0.092 | 52/11 | — |
| 26 | H | CH₂OCH₃ | 0.008 | 6.3 | 0.042 | 48/8 | 3/135/>1000 |
| 27 | H | cBu | 0.005 | 4.9 | 0.020 | 37/13 | 4/58/910 |
| 28 | H | cPent | 0.015 | 4 | 0.145 | 47/11 | — |
| 29 | H | 3-THF | 0.017 | 5.8 | 0.205 | 45/8 | — |
| 30 | H | 3-furyl | 0.005 | 5.3 | 0.104 | — | — |
| 31 | H | OH | >10 | — | — | — | — |
| 32 | H | NMe₂ | >1 | — | — | — | — |

Representative compounds of Formula I include, but are not limited to, the following compounds of Table 4 below, or pharmaceutically acceptable salts thereof

TABLE 4

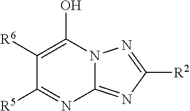

| | | | | CCR9 | | Chemotaxis | RLM/HLM | |
|---|---|---|---|---|---|---|---|---|
| | | | | Ca²⁺ IC₅₀ | Lip | (buffer, | CL | Sol. pH 4/7/9 |
| ID | R² | R⁵ | R⁶ | (uM) | E | uM) | (mL/min/kg) | (ug/mL) |
| 2 | CF₃ | Me | 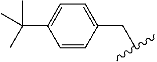 [SMILES: *Cc1ccc(C(C)(C)C)cc1] | 0.41 | 2.6 | — | 54/19 | 4/565/>1000 |
| 33 | CF₃ | Me | 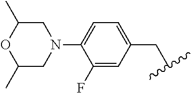 [SMILES: *Cc1ccc(N2CC(C)OC(C)C2)c(F)c1] | 1.9 | 3 | — | —/17 | 79/>1000/>1000 |
| 34 | CF₃ | Me | 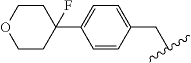 [SMILES: *Cc1ccc(C2(F)CCOCC2)cc1] | 0.34 | 3.7 | — | 43/17 | — |
| 35 | H | Et | 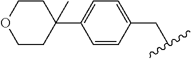 [SMILES: *Cc1ccc(C2(C)CCOCC2)cc1] | 0.022 | 5.7 | 0.38 | 37/17 | >1000/>1000/>1000 |
| 36 | H | Et | 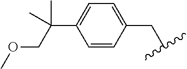 [SMILES: *Cc1ccc(C(C)(C)COC)cc1] | 0.013 | 5.9 | 0.06 | 49/20 | 62/>1000/>1000 |
| 37 | H | Et | 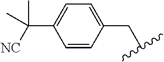 [SMILES: *Cc1ccc(C(C)(C)C#N)cc1] | 0.064 | 5.6 | 0.50 | 7/1 | >1000/994/982 |
| 38 | CH₂OCH₃ | CH₂OCH₃ | 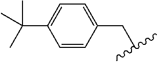 [SMILES: *Cc1ccc(C(C)(C)C)cc1] | 0.013 | 5.9 | 0.20 | 15/5 | 132/999/989 |

Compounds of Formulae I, II, III, IV and V, as well as compounds of Table 1, Table 2, Table 3, Table 4, and Table 5 are also referred to herein as “compounds of the invention.”

Another object of the present invention is the use of a compound as described herein in the manufacture of a medicament for use in the treatment of a disorder or disease herein. Another object of the present invention is the use of a compound as described herein for use in the treatment of a disorder or disease herein.

Another aspect is an isotopically labeled compound of Formulae I, II, III, IV or V delineated herein. Such compounds have one or more isotope atoms which may or may not be radioactive (e.g., ³H, ²H, ¹⁴C, ¹³C, ³⁵S, ³²P, ¹²⁵I, and ¹³¹I) introduced into the compound. Such compounds are useful for drug metabolism studies and diagnostics, as well as therapeutic applications.

Some of the compounds of this invention have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present invention. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). The compounds of this invention may also be represented in multiple tautomeric forms, in such instances the invention expressly includes all tautomeric forms of the compounds described herein. When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present invention. All crystal forms of the compounds described herein are expressly included in the present invention.

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present invention. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In embodiments, the invention provides for the intermediate compounds of the formulae delineated herein and methods of converting such compounds to compounds of the formulae herein (e.g., in schemes herein) comprising reacting a compound herein with one or more reagents in one or more chemical transformations (including those provided herein) to thereby provide the compound of any of the formulae herein or an intermediate compound thereof.

The synthetic methods described herein may also additionally include steps, either before or after any of the steps described in any scheme, to add or remove suitable protecting groups in order to ultimately allow synthesis of the compound of the formulae described herein. The methods delineated herein contemplate converting compounds of one formula to compounds of another formula (e.g., in Exemplification, section I. General synthetic scheme for the synthesis of hydroxypyrimidine triazoles). The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, sublimation, crystallization, trituration, solid phase extraction, and chromatography).

Also disclosed herein are methods for treating inflammatory disease in a subject in need thereof comprising administering to the subject a pharmaceutical composition of a CCR9 inhibitor (i.e., a compound Formulae I, II, III, IV and V). Thus, provided herein are methods for treating inflammatory disease in a subject in need thereof comprising administering to the subject a therapeutically effective amount of a CCR9 inhibitor (i.e., a compound Formulae I, II, III, IV and V).

Chemokines and their associated receptors (e.g., TECK and CCR9, respectively) are proinflammatory mediators that promote recruitment and activation of multiple lineages of leukocytes and lymphocytes. Continuous release of chemokines at sites of inflammation mediates the ongoing migration of effector cells in chronic inflammation. CCR9 and its associated chemokine TECK, have been implicated in chronic inflammatory diseases, such as inflammatory bowel diseases. Small molecule inhibitors of the interaction between CCR9 and its ligands (e.g., TECK), such as the compounds provided herein, are useful for inhibiting harmful inflammatory processes triggered by receptor-ligand interactions and thus are useful for treating diseases mediated by CCR9, such as chronic inflammatory diseases.

In one aspect, provided herein is a method of treating an inflammatory disease in a subject in need thereof, comprising administering to the subject an effective amount of a compound of Formula I, provided herein. In one embodiment, the inflammatory disease is inflammatory bowel disease. In another embodiment, the inflammatory disease is Crohn's disease or ulcerative colitis.

In another aspect, provided herein is a method of inhibiting a CCR9 receptor function in a subject in need thereof, comprising the step of administering to the subject an effective amount of a compound of Formula I, provided herein. In one embodiment, the compound inhibits the binding of a ligand to CCR9. In yet another embodiment, the ligand is TECK.

In yet another aspect, provided herein is a method of inhibiting CCR9-mediated homing of leukocytes in a subject in need of such treatment, comprising administering to the subject an effective amount of at least one compound of Formula I, provided herein.

The subject considered herein is typically a human. However, the subject can be any mammal for which treatment is desired. Thus, the methods described herein can be applied to both human and veterinary applications.

In other embodiments, kits are provided. Kits according to the invention include package(s) comprising compounds or compositions of the invention. In some embodiments, kits comprise a compound provided herein, or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) and/or any product insert(s) may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

Listed below are definitions of various terms used to describe this invention. These definitions apply to the terms as they are used throughout this specification and claims, unless otherwise limited in specific instances, either individually or as part of a larger group.

The term “alkyl,” as used herein, refers to saturated, straight- or branched-chain hydrocarbon moieties containing, in certain embodiments, between one and six, or one and eight carbon atoms, respectively. Examples of C₁-C₆ alkyl moieties include, but are not limited to, methyl, ethyl, propyl, isopropyl, n-butyl, tert-butyl, neopentyl, n-hexyl moieties; and examples of C₁-C₈ alkyl moieties include, but are not limited to, methyl, ethyl, propyl, isopropyl, n-butyl, tert-butyl, neopentyl, n-hexyl, heptyl, and octyl moieties.

The number of carbon atoms in a hydrocarbyl substituent can be indicated by the prefix “Cₓ-C_{y},” where x is the minimum and y is the maximum number of carbon atoms in the substituent. Likewise, a Cₓ chain means a hydrocarbyl chain containing x carbon atoms.

The term “alkoxy” refers to an —O-alkyl moiety or an alkyl-O-alkyl moiety.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like. In some embodiments, aryl groups have 6 carbon atoms. In some embodiments, aryl groups have from six to ten carbon atoms. In some embodiments, aryl groups have from six to sixteen carbon atoms. The term “aralkyl,” or “arylalkyl,” as used herein, refers to an alkyl residue attached to an aryl ring. Examples include, but are not limited to, benzyl, phenethyl and the like.

The term “carbocyclic,” as used herein, denotes a monovalent group derived from a monocyclic or polycyclic saturated, partially unsatured, or fully unsaturated carbocyclic ring compound. Examples of carbocyclic groups include groups found in the cycloalkyl definition and aryl definition.

The term “cycloalkyl,” as used herein, denotes a monovalent group derived from a monocyclic or polycyclic saturated or partially unsatured carbocyclic ring compound. Examples of C₃-C₈-cycloalkyl include, but not limited to, cyclopropyl, cyclobutyl, cyclopentyl, cyclohexyl, cycloheptyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heterocycle” or “heterocyclyl” refers to a five-member to ten-member, fully saturated or partially unsaturated nonaromatic heterocylic groups containing at least one heteroatom such as O, S or N. The most frequent examples are piperidinyl, morpholinyl, piperazinyl, pyrrolidinyl or pirazinyl. Attachment of a heterocyclyl substituent can occur via a carbon atom or via a heteroatom.

The term “halo” as used herein, refers to an atom selected from fluorine, chlorine, bromine and iodine.

The term “haloalkyl,” as used herein, refers to an alkyl moiety substituted with one or more atoms selected from fluorine, chlorine, bromine and iodine.

The term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Additionally, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

The terms “treating” or “treatment” indicates that the method has, at the least, mitigated inflammation. For example, the method can reduce the rate of inflammation in a patient, or prevent the continued inflammation, or even reduce the overall reach of the inflammation. In another embodiment, the terms “treating” or “treatment” can refer to any improvement in one or more clinical symptoms of an inflammatory disease.

### Examples

### I. Synthesis

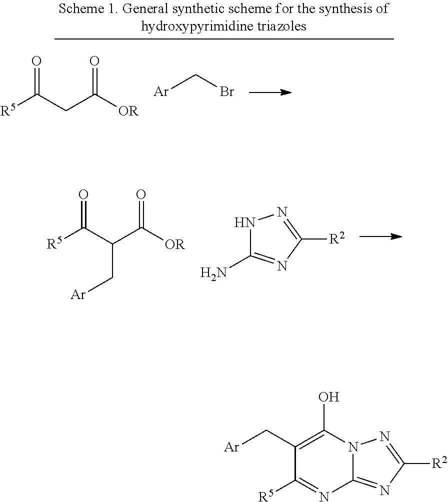

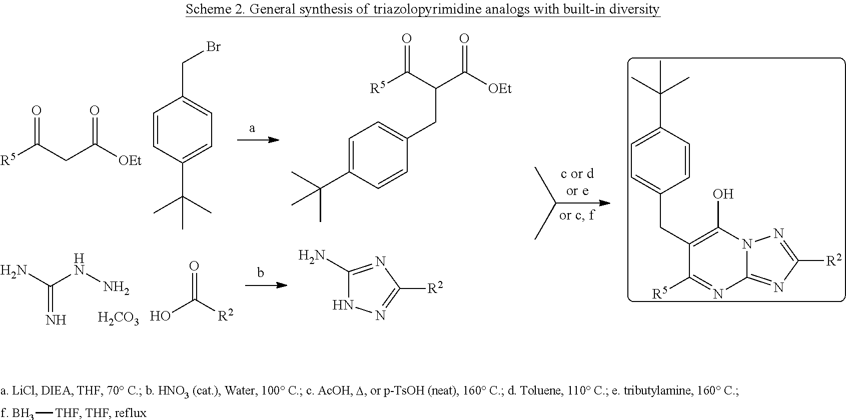

### Synthetic Methods for Representative Scheme 2

β-Keto esters were alkylated in moderate to excellent yields, and subsequently cyclized to introduce benzylic tails at the R⁶ position of the triazolopyrimidine core.

Diversity at the R² position was achieved through triazole cyclizations with aminoguanidine and functionalized carboxylic acids.

Alkylated β-keto esters underwent pyrimidine cyclizations with corresponding aminotriazoles under acidic, neutral, or basic conditions to access triazolopyrimidinols.

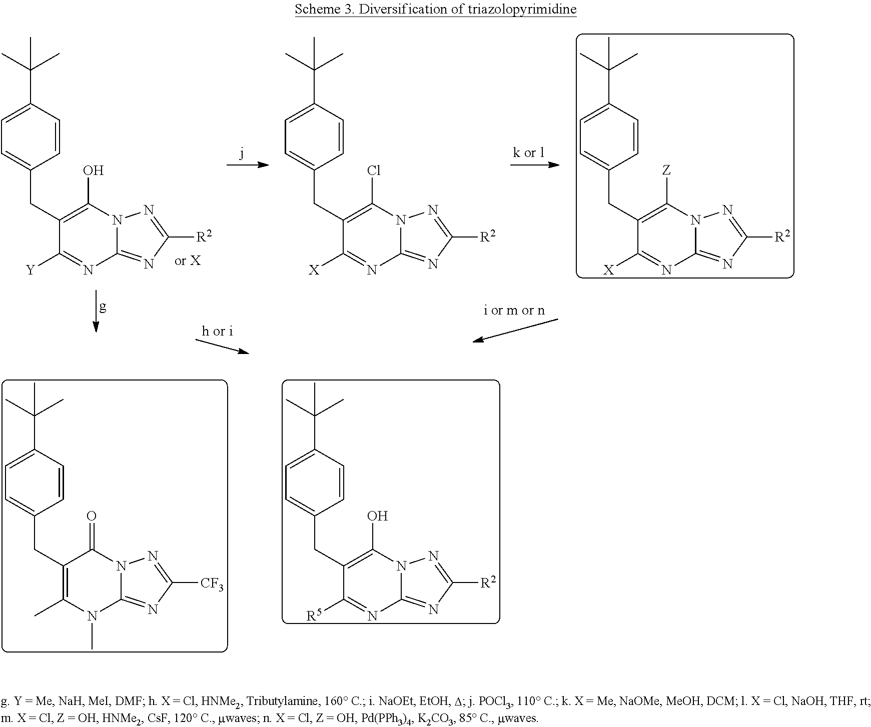

Synthetic methods for diversification according to Scheme 3. Functionalized triazolopyrimidinol analogs were selectively alkylated at the N-4 position.

Halogenated R² (X) analogs were subjected to displacement reaction to achieve N- and O-linked chains.

Chlorination of hydroxyl groups on the pyrimidine ring provided access to N- and O-linked modifications, as well as aryl groups through Suzuki couplings.

TABLE 5
Synthetic details and yields of synthesized analogs

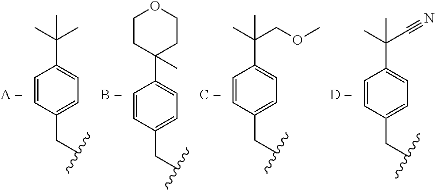 [SMILES: *Cc1ccc(C(C)(C)C)cc1 ; *Cc1ccc(C2(C)CCOCC2)cc1 ; *Cc1ccc(C(C)(C)COC)cc1 ; *Cc1ccc(C(C)(C)C#N)cc1]

| | | Synthetic | Yield |
|---|---|---|---|
| ID | Structure | Sequence | (%) |
| 2 | 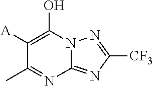 | a, d | a = 73, d = 62 |
| 3 | 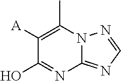 | a,d | d = 16 |
| 4 | 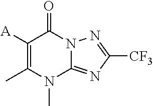 | a, d, g | g = 48 |
| 5 | 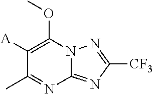 | a, d, j, k | j = 93, k = 33 |
| 7 | 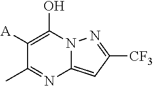 | a, c | c = 67 |
| 11 | 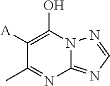 | a, c | c = 67 |
| 12 | 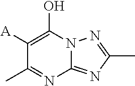 | a, c | c = 36 |
| 13 | 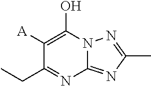 | a, c | a = 61, c = 9 |
| 14 | 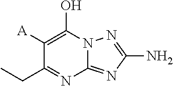 | a, c | c = 19 |
| 15 | 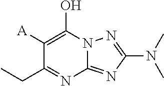 | a, c, h | c = 39, h = 74 |
| 16 | 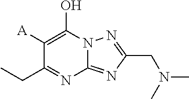 | a, c, f | c = 8, f = 11 |
| 18 | 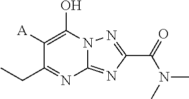 | a, c | c = 8 |
| 19 | 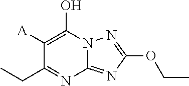 | a, c, i | c = 63, i = 26 |
| 17 | 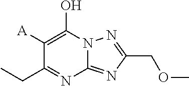 | a, c | c = 26 |
| 20 | 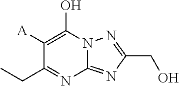 | a, c | c = 4 |
| 21 | 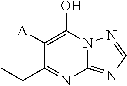 | a, c | c = 35 |
| 22 | 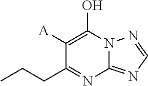 | a, c | a = 50, c = 7 |
| 23 | 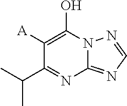 | a, c | a = 50, c = 11 |
| 25 | 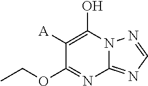 | a, c, j, l, i | j = 77, l = 84, i = 10 |
| 26 | 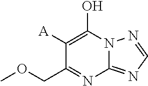 | a, c | a = 90, c = 65 |
| 27 | 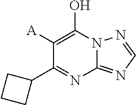 | a, c | a = 92, c = 17 |
| 28 | 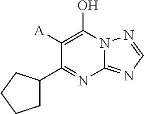 | a, c | a = 74 c = 12 |
| 29 | 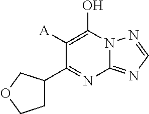 | a, e | a = 40 e = 17 |
| 30 | 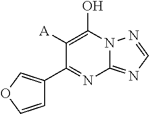 | a, e, j, l, n | n = 33 |
| 31 | 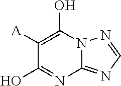 | a, e | a = 59, e = 33 |
| 32 | 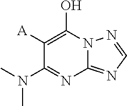 | a, c, j, l, m | m = 3 |
| 38 | 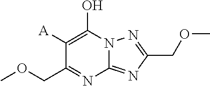 | a, b, c | c = 37 |
| 35 | 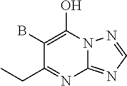 | a, c | a = 27, c = 7 |
| 36 | 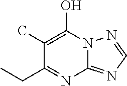 | a, c | a = 50, c = 14 |
| 37 | 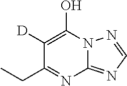 | a, c | a = 68, c = 28 |
| 39 | 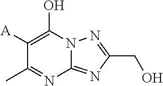 | | |
| 40 | 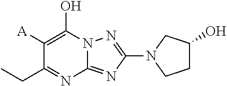 | | |
| 41 | 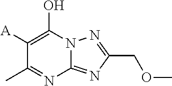 | | |
| 42 | 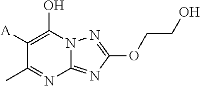 | | |

### β-Keto Ester Intermediates

### Ethyl 2-(4-(tert-butyl)benzyl)-3-oxobutanoate

N,N-Diisopropylethylamine (17 ml, 98 mmol, 2.0 eq) was added to lithium chloride (2.1 g, 49 mmol, 1.0 eq), ethyl acetoacetate (6.2 ml, 49 mmol, 1.0 eq), and 4-(tert-butyl)benzyl bromide (9 ml, 11 g, 49 mmol, 1.0 eq) in 100 mL THF. The mixture was stirred at 80° C. for 12 h. The reaction mixture was cooled to room temperature and partitioned between ethyl acetate and water. The layers were separated and the aqueous layer was and extracted 3×20 mL EtOAc. The organic layers were combined, washed with brine, dried with sodium sulfate, filtered, and concentrated. The resulting oil was purified by silica gel chromatography (0-10% ethyl acetate:hexanes) to yield a clear and colorless oil (7.7 g, 28 mmol, 57%).

¹H NMR (400 MHz, Chloroform-d) δ 7.31 (d, J=8.0 Hz, 2H), 7.13 (d, J=8.0 Hz, 2H), 4.18 (q, J=7.0 Hz, 2H), 3.79 (t, J=7.6 Hz, 1H), 3.16 (d, J=7.6 Hz, 2H), 2.22 (s, 3H), 1.32 (s, 9H), 1.22 (t, J=7.1 Hz, 3H).

### Methyl 2-(4-(tert-butyl)benzyl)-3-oxopentanoate

¹H NMR (400 MHz, Chloroform-d) δ 7.31 (d, J=8.1 Hz, 2H), 7.11 (d, J=8.0 Hz, 2H), 3.82 (t, J=7.5 Hz, 1H), 3.72 (s, 3H), 3.16 (d, J=7.5 Hz, 2H), 2.66-2.52 (m, 1H), 2.45-2.29 (m, 1H), 1.31 (s, 9H), 1.02 (t, J=7.2 Hz, 3H).

### ethyl 2-(4-(tert-butyl)benzyl)-4-methoxy-3-oxobutanoate

¹H NMR (400 MHz, Chloroform-d) δ 7.31 (d, J=7.7 Hz, 2H), 7.13 (d, J=7.7 Hz, 2H), 4.17 (q, J=7.1 Hz, 2H), 4.08 (d, J=17.3 Hz, 1H), 3.97-3.84 (m, 2H), 3.33 (s, 3H), 3.18 (m, 2H), 1.31 (s, 9H), 1.22 (t, J=7.1 Hz, 3H).

### Hydroxypyrimidinetriazoles

Compound 2:

To a solution of ethyl 2-(4-(tert-butyl)benzyl)-3-oxobutanoate (0.20 g, 0.72 mmol) dissolved in 2.4 mL toluene was added 3-(trifluoromethyl)-1H-1,2,4-triazol-5-amine (0.11 g, 0.72 mmol). The solution was heated to 110° C. for 40 h. After completion of the reaction, the reaction was cooled to room temperature, concentrated in vacuo, and purified by silica gel chromatography (0-50% ethyl acetate:hexanes) to yield the product as a white solid (0.16 g, 0.45 mmol, 62%). ¹H NMR (400 MHz, DMSO-d₆) δ 13.55 (s, 1H), 7.27 (d, J=8.1 Hz, 2H), 7.17 (d, J=8.1 Hz, 2H), 3.84 (s, 2H), 2.38 (s, 3H), 1.24 (s, 9H).

Compound 11:

Ethyl 2-(4-(tert-butyl)benzyl)-3-oxobutanoate (0.2 g, 0.71 mmol), p-toluenesulfonic acid monohydrate (0.14 g, 0.71 mmol), and 1H-1,2,4-triazol-5-amine (0.060 g, 0.71 mmol) were added to a 5 mL microwave vial. The microwave vial was sealed, heated to 160° C., and stirred for 40 h as a melt reaction. The residue was dissolved in a minimal amount of solvents and purified by reverse phase HPLC (acetonitrile:water: 0.1% formic acid as eluent) to yield the product as a white solid (0.14 g, 0.48 mmol, 67%). ¹H NMR (400 MHz, DMSO-d₆) δ 13.15 (s, 1H), 8.19 (d, J=4.1 Hz, 1H), 7.21 (dd, J=47.1, 8.1 Hz, 4H), 3.81 (s, 2H), 2.35 (d, J=4.1 Hz, 3H), 1.24 (d, J=4.1 Hz, 9H).

Compound 38:

Ethyl 2-(4-(tert-butyl)benzyl)-4-methoxy-3-oxobutanoate (0.34 g, 1.1 mmol, 1.0 eq) was added to a solution of 5-(methoxymethyl)-4H-1,2,4-triazol-3-amine (0.28 g, 2.2 mmol, 2.0 eq) in acetic acid (1.1 mL). The reaction was stirred at 120° C. for 18 h. The solvent was removed in vacuo. The residue was purified by flash chromatography on silica gel (0-5% methanol: dichloromethane) to yield the product 6-(4-(tert-butyl)benzyl)-2,7-bis(methoxymethyl)-[1,2,4]triazolo[1,5-a]pyrimidin-5-ol (0.15 g, 0.41 mmol, 37%).

¹H NMR (400 MHz, DMSO-d₆) δ 13.14 (s, 1H), 7.27 (d, J=7.8 Hz, 2H), 7.16 (d, J=8.0 Hz, 2H), 4.50 (s, 4H), 3.86 (s, 2H), 3.34 (d, J=5.9 Hz, 6H), 1.24 (s, 9H).

### Identification and Development of Small Molecule Inhibitors of CCR9

Inflammatory bowel disease (IBD), including Crohn's disease (CD) and ulcerative colitis (UC), is characterized by chronic inflammation in the gastrointestinal (GI) tract with periods of flare and remission. Steroids and immuno-modulators are widely used treatments but are sub-optimal in their effectiveness. In the last decade, the standard of care in the US has become anti-TNFα monoclonal antibodies (e.g. infliximab) in combination with the immunosuppressant azathioprine which benefits two thirds of CD patients. Of the patients that do respond, 50% remain in remission after a year of therapy. Thus, there remains a significant unmet medical need for those that either did not respond initially or did not maintain their remission after one year.

The GI tract inflammation associated with IBD results from inappropriate recruitment and accumulation of leukocytes in the gut. CCR9 is a key mediator for pro-inflammatory T cells to migrate from the blood stream to the gut tissue. The CCR9 ligand (CCL25) is expressed predominantly in the thymus and the small intestine. In CD patients, chemokine CCL25 is overexpressed in the small intestine and CCR9+ lymphocytes are reported to be significantly elevated. Given the biological link of CCR9, CCL25, and gut inflammation, we set out to develop CCR9 antagonists for the treatment of IBD.

CCR9 (Chemokine Receptor) antagonists are considered a viable target for treatment of Intestinal Bowel Syndrome and Crohn's Disease. Medicinal Chemistry efforts focused on identifying potent small-molecule CCR9 antagonists yielded an acidic triazole series in hit-to-lead identification. However, during early ADME characterization, the acidic triazoles demonstrated high variability in their oral absorption (i.e. Cₘₐₓ, Tₘₐₓ, AUC and biphasic absorption profiles) and high metabolic clearance. Absorption modeling (GASTROPLUS) was performed on selected acidic triazole compounds to guide formulation and chemical modifications. In addition, an in vitro microsomal model was developed to drive the SAR for improved in vivo clearance. In combination, the two approaches resulted in significant absorption and clearance improvements and provided new triazole analogs with consistent oral absorption and extended exposure.

### High Throughput Screening

Compound Library and Screening:

A screening campaign of 357,199 compounds from diverse libraries was conducted by Evotec (Hamburg, Germany) to identify small molecule antagonists of CCR9. The screen was performed using a FLIPR based assay in 384 well assay plates. The primary screen was done for antagonists at 20 μM in singlicate. 24,832 primary active compounds were identified and 4,620 were selected for confirmation by testing in triplicate under identical conditions as for the primary screen. IC₅₀s against CCR9 were determined for 535 of the 2,056 confirmed hits using an 11-point dose-response curve with top concentration of 40 μM. In order to identify assay artifacts and non-specific compounds, selected compounds were profiled using selectivity assays for the PAR and M1 receptors in a similar format to CCR9 and an orthogonal PathHunter β-Arrestin assay.

Data Processing and Analysis:

Screening hit selection was achieved based on activity against CCR9, physical-chemical properties, and structure diversity. Multiple statistical methods were used to scale and score compounds with a bias against sulphonamide chemotypes. This included a voting based scoring system using percent inhibition, combined with position corrections based on cell, row and column position, and structural similarity to the other hits. The confirmed compounds were automatically scored for lead-likeness and manually inspected before profiling. All active compounds identified were checked for purity before being analyzed for potential structure-activity relationship. Active compound classes were prioritized based on their activity against CCR9, ligand efficiency and preliminary SAR.

### Hit Identification, Analysis and SAR by Catalog

Pyrimidone 1 emerged from the HTS as a selective and potent antagonist of CCR9. Activity of the compound was confirmed in our primary calcium mobilization (Ca FLIPR) assay and an orthogonal GTPγS assay. Further analysis of the structure suggests that the pyrimidinone motif can also exist as the tautomeric hydroxypyrimidine. The measured pKa of Pyrimidone 1 suggests that at physiological pH, the analogs can exist as charged hydroxypyrimidines.

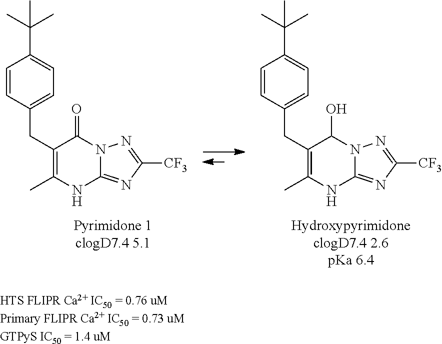 [SMILES: Cc1[nH]c2nc(C(F)(F)F)nn2c(=O)c1Cc1ccc(C(C)(C)C)cc1 ; CC1=C(Cc2ccc(C(C)(C)C)cc2)C(O)n2nc(C(F)(F)F)nc2N1]

As a follow-up to compound 1, our initial approach focused on an SAR by catalog effort to validate the series. Favorable SAR from 36 commercially available analogs (BIONET/Key Organics) provided additional support for medicinal chemistry prioritization of the triazole series.

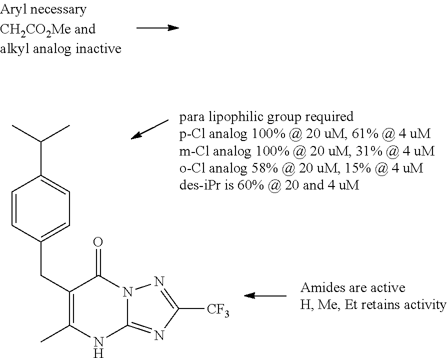 [SMILES: Cc1[nH]c2nc(C(F)(F)F)nn2c(=O)c1Cc1ccc(C(C)C)cc1]

### Assay Methods

Calcium Mobilization Assay:

Cells expressing CCR9 receptor (MultiSpan, Hayward, Calif.) were seeded in 384-well plates. Ca²⁺ assays were conducted after overnight culture in the plates according to the manufacturer's protocol using Screen Quest™ Fluo-8 no wash kit (AAT Bioquest, Sunnyvale, Calif.). Dye loading buffer was added to the cells and incubated for 45 minutes at 37° C. followed by 15 minutes incubation at room temperature. Compounds in the presence of 0.1% DMSO were applied to the cells during calcium flux measurement. Calcium flux was monitored for 90 seconds with compound application after 10 seconds. For antagonist mode, cells were preincubated with the compound at room temperature for 10 minutes before the application of the control agonist TECK (Preprotech, Rocky Hill, N.J.) at EC80 concentration of 0.01 uM obtained from dose-response curves of control agonist.

Chemotaxis Assay:

Molt-4 cells were harvested and re-suspended in HBSS buffer with 0.1% BSA or in 100% human serum. Cell suspensions were mixed with compound solutions for 10 min and then seeded onto the upper chamber of the ChemoTX chemotaxis plates with 5 μm pore size polycarbonate membrane from NeuroProbe (Gaithersburg, Md.). EC80 concentration of TECK was applied in the bottom chamber. After 2 hours of incubation at 37° C., the assay was terminated by removing the upper chamber. The cells migrated into the bottom chamber were quantified with CyQUANT solution from Invitrogen (Grand Island, N.Y.).

In-Silico Modeling:

Parameter Sensitivity Analysis (PSA) was performed using GASTROPLUS simulation software in order to determine the sensitivity of oral bioavailability of acidic triazoles to their physicochemical properties. Experimental PK data (time, concentration and % CV) from i.v. and p.o. dosing in rat were entered into the PKPlus module and a best fit compartmental model was determined based on R² values. The resulting CL, Vc, and rate constants were exported into the drug record and simulations of p.o. concentration vs. time profiles were performed and compared to experimental results.

pH-Solubility:

Solubility of representative acidic triazoles was measured in 3 buffer systems (pH 4, 7.4 and 9) using an in-house high-throughput solubility method. A stock solution of test article was prepared in DMA (20 mg/ml) and spiked into each buffer system (20× dilution) to target 1 mg/ml concentration. The samples were vortexed briefly and equilibrated for 24 hr at RT. Samples were then filtered using 0.22u PVDF membrane filters and analyzed for drug concentration using HPLC.

Formulation:

Dosing solutions at 1 mg/mL for rat PK studies were prepared using either a pH+co-solvent approach or pH+cosolvent+surfactant approach. The total amounts of cosolvent and surfactant used in the solution formulations ranged from 5-8% v/v and q.s. with pH 9 phosphate buffer. Solutions were assessed for their potential for in-vivo precipitation using an in-vitro dilution test (1:1, 1:4 and 1:9) in simulated gastric and intestinal fluids.

Microsomal In-Vitro Clearance:

The test articles (2 μM) were incubated for one hour in 1 mg/mL rat hepatic microsomes with 2 mM NADPH at 37° C. with aliquots removed at t=0, 15, 30 and 60 minutes. Incubations were terminated at the specified times by protein precipitation, and samples were centrifuged. Resultant supernants were analyzed by LC-MS/MS for the amount of incubated compound remaining, and the half life (t_{1/2}) and intrinsic microsomal clearance rate (CLᵢₙₜ) of each compound was calculated.

Plasma and Microsome Protein Binding:

Protein binding was measured via high throughput equilibrium dialysis with the HTDialysis device fitted with a 12,000 to 14,000 Da molecular weight cutoff membrane. Rat plasma or 1 mg/mL rat hepatic microsomes spiked with test article were dialyzed vs. phosphate buffer for 6 hours. Aliquots were removed from both sides of the membrane and diluted with equal volumes of the opposite matrix. Following protein precipitation and centrifugation, the resultant matrix matched supernatants were analyzed by LC-MS to compare the plasma or microsome dialysate test article signal to the buffer signal. Test article stability was monitored during the course of the assay.

Rat PK (i.v./p.o):

Sprague Dawley Rats (n=3) were dosed both intravenously and orally with a solution formulation followed by plasma collection at time points providing sufficient coverage of the absorption, distribution, metabolism and excretion phases of the test article. Test article concentrations at each time point were determined by LC-MS detected bioanalytical analysis. Non-compartmental pharmacokinetic analysis of the intravenous bioanalytical data provided area under the curve, clearance, volume of distribution, and terminal half life parameters while similar analysis of plasma samples from animals dosed orally provided area under the curve, bio-available fraction and oral terminal half life pharmacokinetic parameters.

### In Vitro Activity Results

Compounds 1-10 and 39-42:

In vitro activities of compounds 1-10 and 39-42 are included in table 1 presented earlier. It can be seen that OH is important for CCR9 antagonist activity. In general, pyrazolo and imidazole analogs lacking a hydrogen bond acceptor are less potent than corresponding triazole analogs. Triazolone analogs probe the importance of hydrogen bond acceptor.

Compounds 11-20:

In vitro activities of compounds 11-20 are included in table 2 presented earlier. It can be seen that various functionality and multiple chemotypes were tolerated. Substituent R² provides an opportunity to improve solubility and tune physicochemical properties, but had little impact on potency.

Compounds 11 and 21-32:

In vitro activities of compounds 21-32 are included in table 3 presented earlier. It can be seen that R⁵ modifications (extension, branching and heteroatom) drives activity for the series, as shown by the significant enhancement of Ca FLIPR IC₅₀ and LipE. Chemotaxis activity improves with more potent Ca IC₅₀, although activity in serum is lacking (data not shown).

Compounds 2 and 33-38:

In vitro activities of compounds 33-38 are included in table 4 presented earlier. It can be seen that modifications to the tert-butyl group resulted in improved in vitro clearance and solubility. R², R⁵, R⁶-modified triazole analogs provided potent compounds with good solubility and in vitro microsomal clearance.

### Pharmacokinetic Profile of Selected Analogs

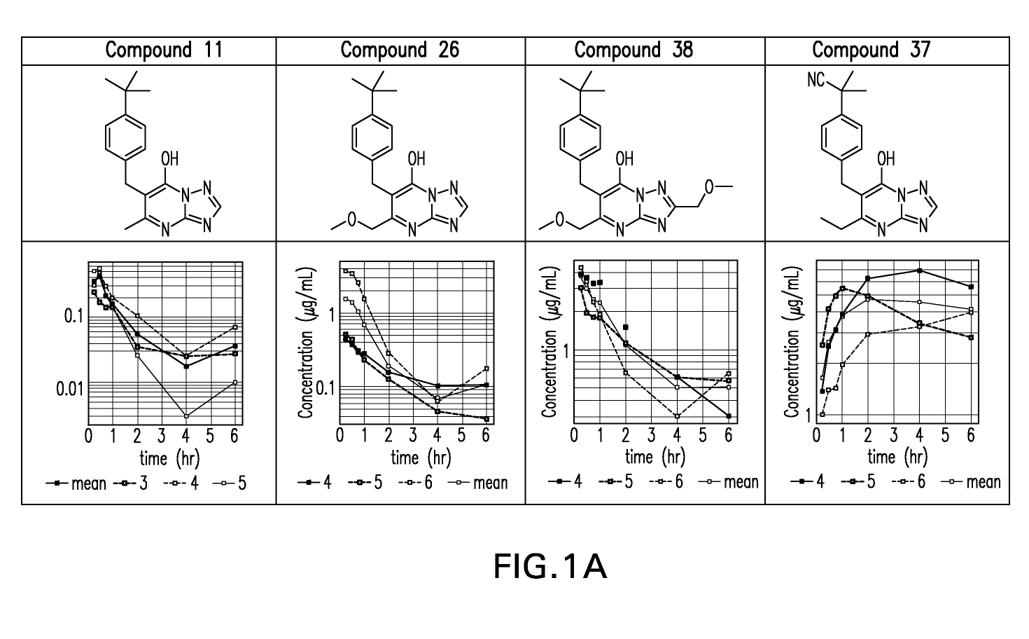

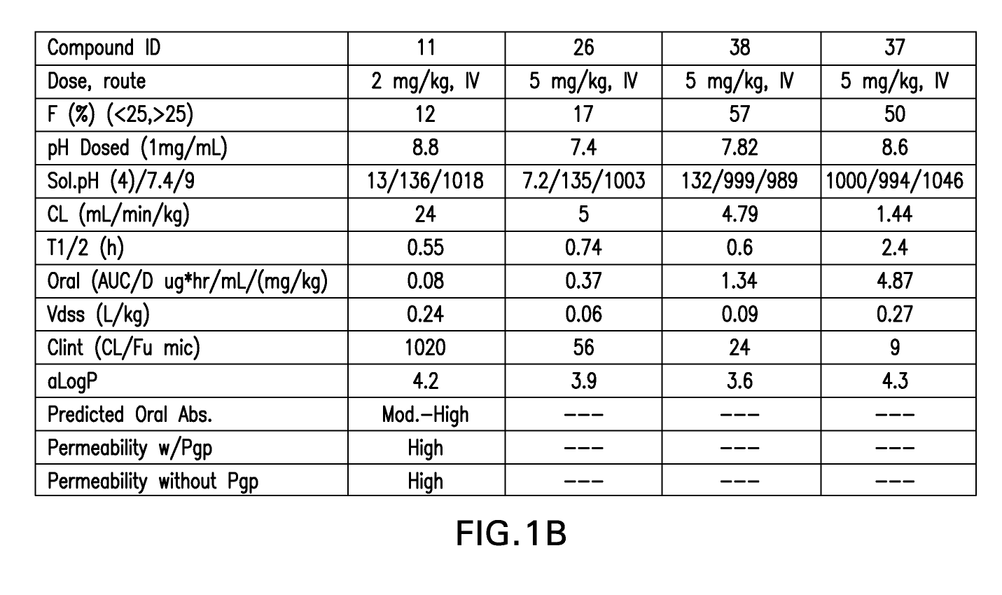

Rats (n of 3) were dosed both intravenously and orally with the various compounds. Plasma samples were collected at time points chosen to provide sufficient coverage of the absorption, distribution, metabolism and excretion phases of the test compound. LC-MS detected bioanalytical analysis was performed on the plasma samples utilizing a standard curve and quality control samples to provide enough precision and accuracy to determine the concentrations test article for plasma from each time point. Non-compartmental pharmacokinetic analysis of the intravenous bioanalytical data provided area under the curve, clearance, volume of distribution, terminal half life parameters while similar analysis of plasma samples from animals dosed orally provided area under the curve, bio-available fraction and oral terminal half life pharmacokinetic parameters (see FIG. 1).

Male rats were dosed as indicated. Colored lines represent IV PK curves of individual animals or mean IV PK as indicated in legends above. Compound 11 had poor PK characteristics, with low oral bioavailability, high in vivo CL, moderate Vd, and short half-life.

Compound 38 has improved solubility and metabolic stability and provides relatively low clearance and good oral exposure, however the Vd remains low.

Compound 37 has lower in vivo CL and increased Vd consistent with increased metabolic stability and extended half-life to provide the highest exposure for the series.

### Oral Bioavailability

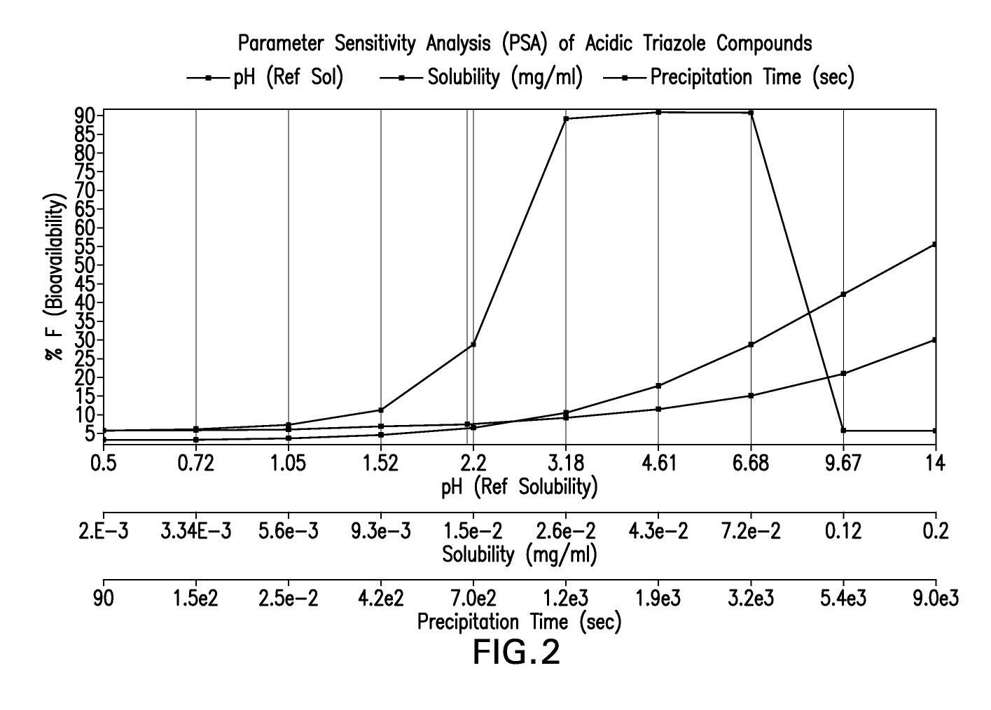

As shown in FIG. 2, the oral bioavailability of acidic triazoles was found to be highly sensitive to their physicochemical properties such as pH (Ref Solubility, pH units), solubility (mg/ml) and precipitation time (sec). Based on the PSA plot, it was predicted that an increase in solubility and precipitation time, especially in the physiological small intestine pH range (4-7), could provide significant improvement in oral absorption and bioavailability of the acidic triazoles and potentially eliminate the highly variable, bi-phasic absorption profiles observed in rat p.o. PK studies. Two different approaches were subsequently taken to improve oral bioavailability of acidic triazoles: (1) formulation modification to extend precipitation time; and (2) chemical modification to improve solubility.

Table 6 highlights the improvement in oral bioavailability achieved with the two approaches as listed above. Compound 6 and compound 39 are two acidic triazoles with similar pH-dependent solubility profile. However, by incorporating a surfactant-based excipient in the oral dosing solution for compound 39, approximately 4-fold increase in its oral bioavailability was achieved. This is most likely due to the delayed in-vivo precipitation of compound 39, as predicted by the PSA plot (FIG. 2) and the in-vitro precipitation time assessment in simulated gastric and intestinal fluids (Table 6).

Compound 40 and compound 42 demonstrate another pair of acidic triazoles where the poorly soluble analog, compound 40, was chemically modified to achieve compound 42 with significantly higher solubility at pH 4 (˜7-fold increase) and pH 7.4 (˜30-fold increase) (Table 6). As a result of this modification and in accordance with the prediction from PSA plot (FIG. 2), a 3-fold increase in oral bioavailability was observed for compound 42 as compared to compound 40.

TABLE 6
Physicochemical and Biopharmaceutical properties of representative Acidic Triazoles

| Compound | 6 | 39 | 40 | 42 |
|---|---|---|---|---|
| Solubility (mg/ml) pH | 0.013/0.14/1 | 0.024/0.26/1 | 0.007/0.02/0.52 | 0.045/0.61/1 |
| 4/7.4/9 | | | | |
| Formulation for Oral PK | Co-solvent | Co-solvent + | Co-solvent + Surfactant (pH 9) | |
| | (pH 9) | Surfactant (pH 9) | | |
| In-vitro Precipitation Time | SGF: 0 min | SGF: 60 min | Not determined | |
| (Visual assessment) | SIF: 120 min | SIF: >240 min | | |
| % F (p.o.) | 12 | 43 | 13 | 43 |
| Oral AUC/D | 0.08 | 0.32 | 0.31 | 1.25 |
| (ug · hr/ml/(mg/kg) | | | | |

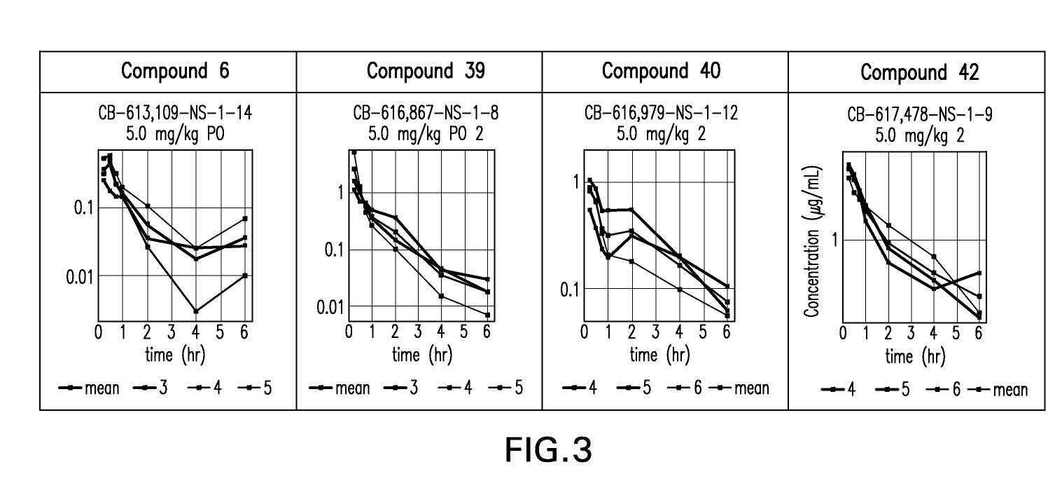

In addition to the improved oral bioavailability, for both instances, the highly variable and bi-phasic absorption profiles were transformed into consistent absorption profiles as highlighted in FIG. 3.

### Metabolic Clearance

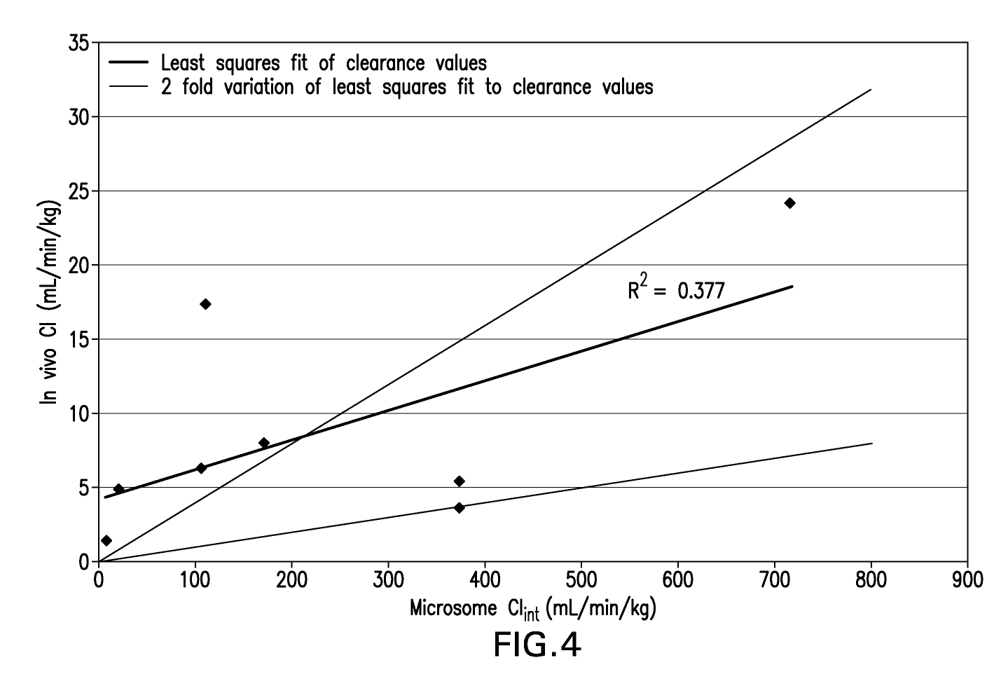

To identify metabolic clearance structure activity relationships in the triazole series the in vivo clearance was compared to the corresponding in vitro microsomal intrinsic clearance and the correlation as found to be poor, R²=0.38 (Table 7 and FIG. 4).

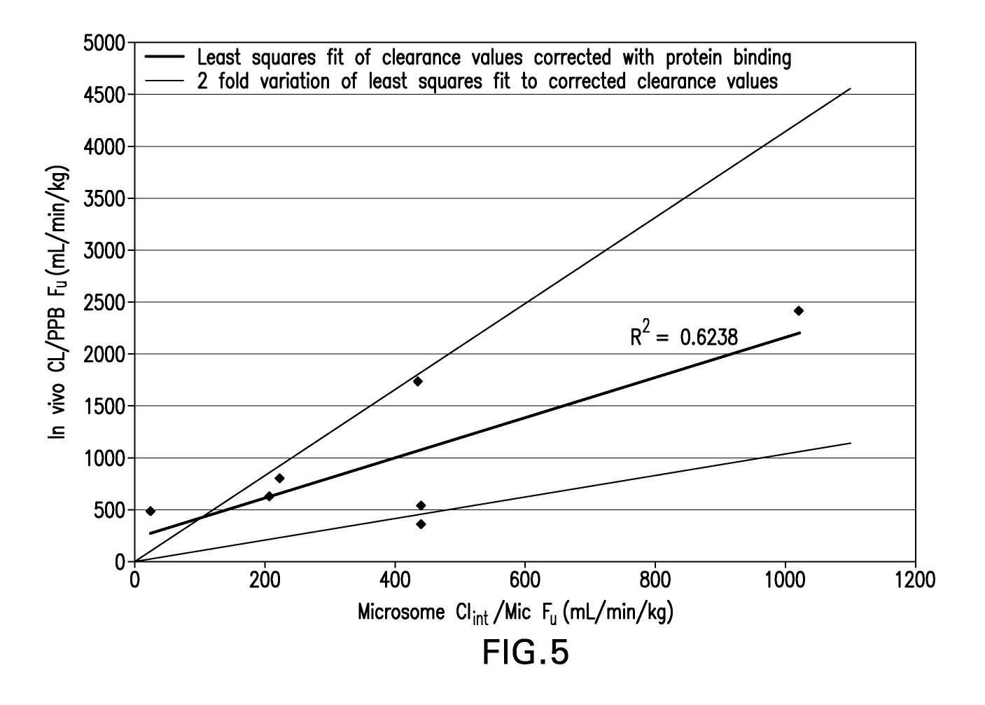

The in vivo-in vitro clearance model was improved to R²=0.62 by incorporating the compounds plasma and microsomal protein binding data with the respective in vivo and in vitro clearance measurements. In this new correlation the compounds fall within approximately two fold of the least squares fit of the protein binding corrected in vivo in vitro clearance data (FIG. 5).

The greatest driver for correcting the in vivo to in vitro correlation came from the dynamic range of the of the microsomal protein binding across the triazole series with microsome Fᵤ ranging from 0.255 to 0.850. In contrast plasma protein binding range was essentially constant at Fᵤ 0.01.

The ability to predict the in vivo beta elimination rate helped guide the chemistry towards compounds with lower in vivo clearance and greater plasma exposure by use of microsome clearance and protein binding data.

Combined application of predictive absorption and clearance models increased the bioavailability and exposure of compounds by increasing Fₐ% and reducing clearance from >40% of hepatic blood flow (HBF) to <10% of HBF.

TABLE 7
Rat Triazole IVIVc, corrected for plasma and microsome protein
binding for in vivo CL and microsome CLᵢₙₜ, respectively

| | | Rat | | | | | |
|---|---|---|---|---|---|---|---|
| | | Plasma | | Rat | | | Rat In |
| | Rat In | Protein | Rat In- | Microsome | Rat | Rat | vivo CL |
| | vivo CL | Binding | vivo CL/ | In Vitro CLᵢₙₜ | Microsomal | Microsome Clint/ | as % Hepatic |
| Compound | (mL/min/kg) | (PPB) Fᵤ | PPB Fᵤ | (mL/min/kg) | Binding Fᵤ | Microsome Fᵤ | Blood Flow |
| 37 | 1 | 0.011 | 131 | 8 | 0.850 | 9 | 3 |
| 6 | 24 | 0.010 | 2420 | 717 | 0.703 | 1020 | 44 |
| 27 | 17 | 0.010 | 1740 | 111 | 0.255 | 435 | 32 |
| 41 | 8 | 0.010 | 800 | 171 | 0.769 | 222 | 15 |
| 40 | 6 | 0.010 | 630 | 106 | 0.513 | 207 | 11 |
| 38 | 5 | 0.010 | 487 | 21 | 0.864 | 24 | 9 |
| 26 | 5 | 0.010 | 540 | 373 | 0.850 | 439 | 10 |
| 42 | 4 | 0.010 | 360 | 373 | 0.850 | 439 | 7 |

HTS efforts successfully identified numerous CCR9 antagonist leads possessing activity in a PathHunter β-Arrestin assay (DiscoveRx Corporation, Fremont, Calif.) and selectivity against PAR and M1 receptors. Nascent SAR from commercially available analogs revealed a pyrimidotriazole series as a viable lead for hit-to-lead medicinal chemistry efforts. Measured pKa of the pyrimidotriazole compound 11 suggested a significant contribution of the hydroxypyrimidine triazole tautomer to the activity of the compound.

Modifications at R², R⁵, and/or R⁶ of the triazole core provided the opportunity to optimize CCR9 FLIPR (assay was performed at MultiSpan, Hayward, Calif.) potency as well as modulate pH 4/7/9 solubility and in vitro microsomal clearance, which translated well to favorable in vivo PK profiles.

Development and implementation of reliable synthetic routes provided ready access to an array of analogs with diversity in key locations around the core.

Variably absorbed, high clearance compounds from acidic triazole series of CCR9 antagonists were transitioned to consistently absorbed, low clearance compounds via modeling, simulation, early formulation screening and in vitro guided clearance SARs.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of Formula I: 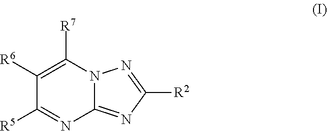
or a pharmaceutically acceptable salt thereof,
wherein:
R² is H, C₁₋₆ alkyl, C₁₋₆ alkoxy optionally substituted one or more times with OH, C₁₋₆ haloalkyl, C₁₋₆ di-haloalkyl, C₁₋₆ tri-haloalkyl, NH₂, N(H)(C₁₋₃ alkyl), N(C₁₋₃ alkyl)₂, (CH₂)₁₋₄—NH₂, (CH₂)₁₋₄—N(H)(C₁₋₃ alkyl), (CH₂)₁₋₄—N(C₁₋₃ alkyl)₂, (CH₂)₁₋₄—C₁₋₆ alkoxy, C(O)NH₂, C(O)N(H)(C₁₋₃ alkyl), C(O)N(C₁₋₃ alkyl)₂, OH, (CH₂)₁₋₄—OH, or a C₃₋₅ heterocycle optionally substituted one or more times with OH;
R⁵ is OH, C₂₋₆ alkyl, C₁₋₆ alkoxy, (CH₂)₁₋₄—C₁₋₆ alkoxy, C₃₋₇ cycloalkyl, N(C₁₋₃ alkyl)₂, or heterocycle;
R⁶ is (CH₂)₁₋₄-aryl, wherein aryl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and
R⁷ is OH, C₁₋₃ alkyl, or C₁₋₃ alkoxy.

2. The compound of claim 1, wherein R² is H, CF₃, or (CH₂)₁₋₄—C₁₋₆ alkoxy.

3. The compound of claim 1 or 2, wherein R² is CF₃ or (CH₂)₁₋₄—C₁₋₆ alkoxy.

4. The compound of any of the above claims, wherein R⁵ is C₂₋₆ alkyl, (CH₂)₁₋₄—C₁₋₆ alkoxy, C₃₋₇ cycloalkyl, heterocycle, OH, NH₂, N(H)(C₁₋₃ alkyl), or N(C₁₋₃ alkyl)₂.

5. The compound of any of the above claims, wherein R⁵ is C₂₋₆ alkyl.

6. The compound of any of the above claims, wherein R⁷ is CH₃ or OH.

7. The compound of any of the above claims, wherein R⁶ is (CH₂)₁₋₄-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy.

8. The compound of any of the above claims, wherein R⁶ is (CH₂)-phenyl, wherein phenyl can be optionally independently substituted one or more times with C₁₋₆ alkyl or C₁₋₆ alkoxy, wherein the C₁₋₆ alkyl group is optionally substituted with CN.

9. The compound of claim 1, or a pharmaceutically acceptable salt thereof, wherein:
R² is H, C₁₋₆ alkyl, CF₃, NH₂, N(H)(C₁₋₃ alkyl), N(C₁₋₃ alkyl)₂, (CH₂)₁₋₄—NH₂, (CH₂)₁₋₄—N(H)(C₁₋₃ alkyl), (CH₂)₁₄—N(C₁₋₃ alkyl)₂, (CH₂)₁₋₄—C₁₋₆ alkoxy, C(O)N(C₁₋₃ alkyl)₂, or (CH₂)₁₋₄—OH;
R⁵ is OH, C₂₋₆ alkyl, C₁₋₆ alkoxy, (CH₂)₁₋₄—C₁₋₆ alkoxy, C₃₋₇ cycloalkyl, or heterocycle;
R⁶ is (CH₂)₁₋₄-phenyl, wherein phenyl can be optionally independently substituted one or more times with C₁₋₆ alkyl, C₁₋₆ alkoxy, halo, or heterocycle, wherein the C₁₋₆ alkyl or heterocycle groups can be optionally independently substituted one or more times with C₁₋₆ alkyl, CN, or C₁₋₆ alkoxy; and
R⁷ is OH.

10. A compound of claim 1, selected from compounds 3, 8, 10, and 40 of Table 1, or pharmaceutically acceptable salts thereof.

11. A compound of claim 1, selected from compounds 13, 14, 15, 16, 17, 18, 19 and 20 of Table 2, or pharmaceutically acceptable salts thereof.

12. A compound of claim 1, selected from compounds 21, 22, 23, 24, 25, 26, 27, 28, 29, 30, 31 and 32 of Table 3, or pharmaceutically acceptable salts thereof.

13. A compound of claim 1, selected from compounds 35, 36, 37 and 38 of Table 4, or pharmaceutically acceptable salts thereof.

14. A pharmaceutical composition, comprising a compound of claim 1 and a pharmaceutically acceptable carrier.

15. A method of treating an inflammatory disease in a subject in need thereof, comprising administering to the subject an effective amount of a compound of claim 1.

16. The method of claim 15, wherein the inflammatory disease is inflammatory bowel disease.

17. The method of claim 15, wherein the inflammatory disease is Crohn's disease or ulcerative colitis.

18. A method of inhibiting CCR9 receptor function in a subject in need thereof, comprising the step of administering to the subject an effective amount of a compound of claim 1.

19. The method of claim 15, wherein the compound inhibits the binding of a ligand to CCR9.

20. The method of claim 19, wherein the ligand is TECK.

21. (canceled)
